(19) United States
(12) Patent Application Publication
(10) Pub. No.: US 2026/0157804 A1
(43) Pub. Date: Jun. 11, 2026

(54) PATELLA TRACKING

(71) Applicant: CAIRA Surgical, New York, NY (US)

(72) Inventors: Morteza MEFTAH, New York, NY (US); Jody Lee CLAYPOOL, Warsaw, IN (US); Gordon L. GOODCHILD, West Palm Harbor, FL (US)

(21) Appl. No.: 19/388,290

(22) Filed: Nov. 13, 2025

Related U.S. Application Data
(60) Provisional application No. 63/719,749, filed on Nov. 13, 2024.

(51) Int. Cl. A61B 34/20

(52) U.S. Cl. CPC A61B 34/20; A61B 2034/2051; A61B 2034/2055; A61B 2034/2072

(57) ABSTRACT

According to one aspect of the invention, a mounting device for mounting a tracking beacon to a patella includes: a beacon mount configured to receive the tracking beacon; and a patella attachment that is configured to attach to an anterior surface of the patella. The patella attachment is configured to attach to the patella such that movement of the patella attachment is fixed relative to the patella, the beacon mount is attached to the patella attachment such that movement of the beacon mount is fixed relative to the patella attachment, and the patella attachment is configured to remain attached to the patella during movement of the patella.

### CROSS-REFERENCE TO RELATED APPLICATION

This application claims the benefit of U.S. Provisional Application No. 63/719,749 filed Nov. 13, 2024, the disclosure of which is hereby incorporated by reference herein in its entirety.

### FIELD

The present technology is generally related to location monitoring of objects in a medical environment.

### BACKGROUND

Placement of implants in bones or soft tissue requires precise planning. For example, in joint replacement orthopedic surgery, precise bony cuts are essential to achieve optimum outcomes. In order to achieve this, historically, manual guides such as cutting blocks that reference boney landmarks, limb anatomical alignment, and visual cues have been designed to help the surgeon perform the cuts; however, these guides lack the necessary precision due to inherent issues with manual cutting jigs.

In recent years, systems and methods utilizing computed assisted surgery (CAS), such as surgical navigation systems and robots, have been developed to improve the accuracy of implant positioning. Existing CAS systems typically require optical trackers for the computer to identify bones that are in constant movement during surgery. These optical trackers include multiple large pins that need to be fixed into each bone, most of the time through separate incisions, which may cause fractures and more pain for the patients. Further, these optical trackers typically need a bulky optical apparatus that requires a line of sight for a camera and large amount of hardware and software in order to operate. Moreover, there is no conventional systematic way to adjust the implant position based on the patients'individual soft-tissue tension. Most CAS systems are configured to achieve a “balanced” soft tissue tension by using surgeons'manual tests. These manual techniques are not accurate or reliably reproducible since the human anatomy varies.

Therefore, existing systems suffer from one or more issues, which may be resolved using radar-based CAS systems, devices, and methods as described herein

### SUMMARY

The techniques of this disclosure generally relate to objection location monitoring in a medical environment.

The techniques of this disclosure include systems and methods for attaching one or more beacons, which may be active, to a bone, for example, a patella, or other part of a patient. In embodiments, the location, orientation, and/or tracking of the bone is performed using radar tracking, optical tracking, or other tracking systems, and thus the beacons may be radar beacons, optical beacons/reflectors, or other types of beacons.

The techniques of this disclosure include a system for the precise tracking of an area of interest comprising one or more radiofrequency (RF) transceivers and one or more active very small beacons that emit radio frequency signals in short-to mid-range distances. These beacons will actively re-transmit the frequency-shifted RF signals (e.g., radar signals) after initial receiving. Since each beacon imposes a unique frequency shift to the incoming radar signal, exhibiting specific Doppler frequencies, the location can be measured with less than one millimeter accuracy. These active beacons are designed to achieve high accuracy, increase the signal to noise ratio, with a small size (less than 1 inch) that can be disposable and also be used with off-the-shelf batteries. Examples of tracking systems are shown, for example, in FIGS. 1 and 2 of U.S. Patent Application Publication No. 2021/0080563 A1, which is incorporated herein by reference in its entirety.

Object triangulation or trilateration: Three primary radars in spaced-apart locations from the region of interest may be used for tracking, for example bone tracking in orthopedic applications and/or tool tracking, (for example a bone saw tool) in orthopedic applications. The three radars emit waves at varying or different frequencies, for example in pulses of milliseconds, so each returning wave will be based on a different transmitted frequency. A calibration device may be used to determine how sub millimeter differences affect each change in distance. In some embodiments, during surgery, a laser range finder may be also attached to the radar(s) to determine and calibrate a true distance from the radar(s) prior to the surgery. Once the ranges are set, wavelengths of appropriate frequency may be used for that range of distance to yield the most accurate readings for the tools and bones. In some embodiments, beacons can also be placed on the radar receivers to detect any changes on the radar locations. Such embodiments may be used to re-calibrate the radars and avoid bias.

In some embodiments, a hand-held scanner (such as a Laser or LIDAR scanner) may be included, which may be tracked by the (e.g., three) radars. This hand-held device may be used to scan the bone surface, e.g. by bouncing light waves from the surface and recording the distance as a laser range finder is again used to find the appropriate wavelength spectrum and to keep track of the scanned areas and how they relate to the new locations as the surgeon moves the device several millimeters or so from and to the joint. This variation may be tracked and all scans may be stitched together to get the true surface geometry. In such embodiments, the scanning device may be tracked in space with the radars similar to the cutting tools to close the loop of bone location determination.

In such embodiments, once the scan is complete, cutting tools such as the free hand bone saw or a cutting block that helps the surgeon make the cuts can be tracked in space and very accurately placed in the appropriate location to achieve the planned surgery.

The scanner can also be used to make measurements after the cuts to determine the accuracy of the cuts to report back to the surgeon to conduct validation.

Tracking the location of the patella poses additional, unusual challenges as compared to many other bones because the patella is a floating bone and, as a result, is subject to more movement during procedures than non-floating bones. Embodiments of this disclosure address these challenges by providing various systems and methods for attaching one or more beacons, (such as active radar beacons or optical beacons), to the patella.

Embodiments provide a device or apparatus that connects to the patella to aid with CAS tracking during knee range of motion procedures, such as CAS tracking during knee arthroplasty, or CAS tracking during diagnosis of patellofemoral maltracking, where the patella tracking can be based on the femoral native anatomy, or CAS tracking after placement of a femoral or trochlear component. In embodiments, the patella apparatus can be used to calculate the patellofemoral compartment during various flexion angles where the patella thickness and the anterior compartment are measured before and after surgery. In embodiments, the patella is first cut and sized, and an appropriate patella plastic button is placed, where the patella has multiple sizes, the patella button undersurface is attached to the bony surface, the patella button has an extension that curves out of the wound and has an attachment for a tracking beacon. In various embodiments, the tracking beacon can be configured for optical tracking, for radar (RF) tracking, or for other types of CAS tracking. In embodiments, the extension is designed to extend distally and then curve outside of the incision so that it has minimum to no interfere with femoral condyles during flexion. In embodiments, the extension can be one piece, attached to the patella button, or modular, such as a ring that is attached between the patella button and the remaining patella bone. In embodiments, the beacon anchor is configured to face a constellation of transceivers (e.g., radar-wave (RF-wave) sources/receivers).

In embodiments where the patella is not resurfaced, the apparatus is attached to the anterior surface of the patella, and the attachment can be configured in a cruciate form. In embodiments, the attachment can be a circular disk with one or more side grips and with a cable that can tighten the grips. In embodiments, the cable can be around the patella. In embodiments, the attachment can be pinned to the anterior cortex of the patella with small hooks, pins, screws, drills, or the like. In embodiments, the beacon attachment can be on the side or center of the disk. In embodiments, the inner surface of the patella can be scanned via a hand-held scanner or the like, and the scanned image data can be superimposed on a prior advanced image, such as an MRI image, a CT scan image, or 3D images from radiograph scanning or ultrasound scanning. In embodiments, the inner surface of the patella can be mapped using a hand-held probe or the like. In embodiments, the CAS tracking system can be optical or RF-based, such as radar-based. In embodiments, the patella tracking, tilt, displacement, and thickness data can be used to plan a virtual osteotomy. This plan can be executed using manual saw, milling, and/or navigated jigs, as well as using a probe and/or a robotic platform.

According to one aspect of the invention, a mounting device for mounting a tracking beacon to a patella includes: a beacon mount configured to receive the tracking beacon; and a patella attachment that is configured to attach to an anterior surface of the patella. The patella attachment is configured to attach to the patella such that the position and/or movement of the patella attachment is fixed relative to the patella, the beacon mount is attached to the patella attachment such that the position and/or movement of the beacon mount is fixed relative to the patella attachment, and the patella attachment is configured to not move relative to, and remain attached to, the patella during movement of the patella.

According to one or more embodiments, the patella attachment includes: a main member that extends across the anterior surface of the patella; a first pin or screw attached to the main member and configured to be inserted into the patella; and a movable second pin or screw attached to the main member and configured to be inserted into the patella.

According to one or more embodiments, the patella attachment includes: a plurality of resilient legs, each of the legs having a hook that is configured to hook onto the patella; a main body having passages configured to receive the legs such that the legs can move within the passages; and a movable member attached to each of the legs. Movement of the movable member causes the legs to move within the passages and move the hooks radially inward relative to the main body.

According to one or more embodiments, the patella attachment includes: a first plate; a first leg fixed to the first plate, the first leg having a hook; a second plate; and a second leg fixed to the second plate, the second leg having a second hook. The first plate is selectably slidable relative to the second plate between a first position and a gripping position, and the first plate and the second plate are configured to be fixed relative to each other such that the first hook of the first leg and the second hook of the second leg grip the patella in the gripping position.

According to one or more embodiments, the patella attachment includes: a plate configured to contact the patella; and a wire-shaped member that is configured to wrap around the patella and attach to the plate to secure the plate to the patella.

According to one or more embodiments, the patella attachment includes: a plate configured to contact the patella; a plurality of sliding members, each of the sliding members having a leg fixed to the sliding member; and an elastic member, which may be wire-shaped, configured to bias the sliding members radially inward such that the legs are biased toward the patella.

According to one or more embodiments, the patella attachment includes: a plate configured to contact the patella; a plurality of sliding members, each of the sliding members having a leg fixed to the sliding member; and a member, which may be wire-shaped, configured to bias the sliding members radially inward such that the legs are biased toward the patella. One of the sliding members is configured to secure the wire-shaped member to the one of the sliding members.

According to one or more embodiments, the patella attachment includes: a plate configured to contact the patella; a plurality of sliding members, each of the sliding members having a leg fixed to the sliding member; a member, which may be wire-shaped, configured to bias the sliding members radially inward such that the legs are biased toward the patella; and a wire-shaped member tensioner configured to apply tension to ends of the wire-shaped member to bias the sliding members radially inward such that the legs are biased toward the patella.

According to one or more embodiments, a system for patella tracking includes: a mounting device of any one of the preceding paragraphs; a plurality of radio frequency transceivers, each of the plurality of radio frequency transceivers configured to emit a radio frequency signal at a first respective frequency; a tracking beacon removably attachable to the mounting device, the tracking beacon being configured to modify the radio frequency signal from the respective first frequency to a second respective frequency different than the first respective frequency; and a control device in communication with the plurality of radio frequency transceivers, the control device including processing circuitry configured to determine a location of the patella in three-dimensional space based at least in part on the modified radio frequency signals for each of the plurality of radio frequency transceivers.

According to one or more embodiments, a mounting device for mounting a tracking beacon to a patella, includes: a beacon mount configured to receive a tracking beacon; a patella attachment that is configured to attach to a resected surface of the patella; and an extension that extends from the patella attachment and attaches the beacon mount to the patella attachment. The beacon mount is attached to the patella attachment via the extension such that a position and/or movement of the beacon mount is fixed relative to the patella attachment, the patella attachment is configured to remain attached to the patella during movement of the patella, and the beacon mount is offset relative to the patella attachment by the extension.

According to one or more embodiments, a system for patella tracking includes: the mounting device of any one of the preceding paragraphs; a plurality of radio frequency transceivers, each of the plurality of radio frequency transceivers configured to emit a radio frequency signal at a first respective frequency; a radio frequency beacon removably attachable to the mounting device, the radio frequency beacon being configured to modify the radio frequency signal from the respective first frequency to a second respective frequency different than the first respective frequency; and a control device in communication with the plurality of radio frequency transceivers, the control device including processing circuitry configured to determine a location of the patella in three-dimensional space based at least in part on the modified radio frequency signals associated with each of the plurality of radio frequency transceivers.

According to one or more embodiments, a system for patella tracking includes: a mounting device; one or more optical fiducial markers attached to the mounting device or to the patella; at least one image sensor configured to capture image frames of the optical fiducial markers during movement of the patella; and a control device including processing circuitry. The processing circuitry may be configured to: (a) identify the optical fiducial markers in the captured image frames using a computer-vision algorithm; and (b) compute a three-dimensional pose of the patella based on the spatial relationship of the optical fiducial markers.

According to one or more embodiments, the optical fiducial markers include passive, non-powered visual patterns. Examples of the passive, non-powered visual patterns include a two-dimensional coded marker, a checkerboard pattern, a color-coded tag, and a geometric feature with known spacing.

According to one or more embodiments, examples of the image sensor include a stereo camera pair, a structured-light depth camera, a time-of-flight sensor, and a handheld scanner with integrated optical tracking.

According to one or more embodiments, the control device is configured to continuously track the patella in six degrees of freedom during knee flexion and extension by processing a sequence of image frames.

According to one or more embodiments, the optical fiducial markers are disposed on an extension that protrudes from the mounting device such that the markers remain in the line-of-sight of the image sensor throughout the knee range of motion.

According to one or more embodiments, the control device is further configured to detect partial or complete occlusion of one or more fiducial markers and to maintain pose estimation through predictive motion modeling or interpolation based on previous image frames.

According to one or more embodiments, the control device is configured to register the tracked patella pose to a pre-operative or intra-operative three-dimensional image of the knee to enable comparison of planned and actual patellar motion.

According to one or more embodiments, the control device employs a trained neural network or other computer-vision model to automatically identify fiducial markers and compute the patella pose with sub-millimeter precision.

According to one or more embodiments, the fiducial markers are detachable and sterilizable, and are configured for re-use across different surgical cases.

According to one or more embodiments, the image sensor is mounted to a surgical instrument, navigation tower, or robotic arm, and the computed patella pose is used to dynamically update a surgical plan or instrument trajectory.

According to one or more embodiments, a method for intraoperative patella tracking includes: (a) attaching a mounting device to the patella; (b) positioning one or more optical fiducial markers on the mounting device; (c) capturing, by at least one image sensor, a sequence of image frames of the fiducial markers as the knee is moved through a range of motion; and (d) determining, by processing circuitry executing a computer-vision algorithm, a three-dimensional pose of the patella from the spatial configuration of the fiducial markers in the captured image frames.

According to one or more embodiments, the method further includes registering the determined patella pose to a pre-operative model or imaging dataset to assess patellofemoral tracking before and after resurfacing or implant placement.

According to one or more embodiments, determining the three-dimensional pose includes triangulating the positions of at least three fiducial markers detected in two or more image frames.

According to one or more embodiments, the method further includes displaying a real-time visualization of patellar tilt, rotation, and translation relative to the femoral component to assist surgical decision-making.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows four possible resection lines of a patella;

FIG. 2 is a perspective view of a beacon mount for tracking movement of a resurfaced patella relative to the femoral groove;

FIG. 3 is a perspective view of FIG. 2 showing the placement of a plastic button;

FIG. 4 is a side view of a patella, femur, and tibia showing a pre-trial patellofermoral offset;

FIG. 5 is a side view of a patella, femur, and tibia showing a post-trial patellofermoral offset including a beacon mount;

FIG. 6 is a side view of a beacon mount on a resurfaced patella;

FIG. 7 is a front view of a beacon mount on a resurface patella showing three pegs of a plastic button attached to the patella;

FIG. 8 is a view from the tibia side of the knee of an exemplary patella griping device using lateral fixation, in accordance with a first embodiment of the disclosure;

FIG. 9 is a view from the front of the knee of the device of FIG. 8;

FIG. 10 is a partial magnified view of FIG. 8;

FIG. 11 is an example of the device of FIG. 8 using oblique fixation on both sides of the patella;

FIG. 12 is a front view of an exemplary patella griping device mounted to a patella using a sizing ring, in accordance with a second embodiment of the disclosure;

FIG. 13 is a side view of the device of FIG. 12 mounted to a patella;

FIG. 14 is an underside view of the device of FIG. 12 mounted to a patella;

FIG. 15 is a front perspective view of the device of FIG. 12 mounted to a patella;

FIG. 16 is a front perspective view of the device of FIG. 12 unattached to a patella;

FIG. 17 is an underside perspective view of the device of FIG. 12 mounted to a patella;

FIG. 18 is a front view of an exemplary patella griping device mounted to a patella using a central tightening screw, in accordance with a third embodiment of the disclosure;

FIG. 19 is a perspective view of the device of FIG. 18 mounted to a patella;

FIG. 20 is a partial magnified view of FIG. 19;

FIG. 21 is a perspective view of the device of FIG. 18;

FIG. 22 is top view of an exemplary patella gripping device in accordance with embodiments of the disclosure;

FIG. 23 is a front view of a knee with the device of FIG. 22 attached to the patella;

FIG. 24 is a front view of a knee with the device of FIG. 22 attached to the patella through soft tissue, and showing a beacon and beacon extension attached to the device;

FIG. 25 is a bottom perspective view of an exemplary patella gripping device in accordance with a fifth embodiment of the disclosure;

FIG. 26 is a top perspective view of an exemplary patella gripping device in accordance with embodiments of the disclosure;

FIG. 27 is a bottom perspective view of the device of FIG. 26;

FIG. 28 is a perspective view of an exemplary patella griping device mounted to a patella using a sliding clamp, in accordance with a fourth embodiment of the disclosure;

FIG. 29 is a perspective view of the device of FIG. 28;

FIG. 30 is front view of the device of FIG. 28;

FIG. 31 is a perspective view of an alternate design of the device of FIG. 28;

FIG. 32 is a perspective view of an exemplary patella griping device mounted to a patella using wire around the patella, in accordance with a fifth embodiment of the disclosure;

FIG. 33 is front view of the device of FIG. 32;

FIG. 34 is a view from the tibia side of the knee of the device of FIG. 32;

FIG. 35 is front view of the device of FIG. 32 with a beacon anchor attached to the device;

FIG. 36 is a perspective view of the device of FIG. 32 with a beacon anchor attached to the device;

FIG. 37 is a front view of the device of FIG. 32 with a beacon anchor attached to the device in a laterally offset position;

FIG. 38 is a perspective view of the device of FIG. 32 with a beacon anchor attached to the device in a laterally offset position;

FIG. 39 is a perspective view of an exemplary patella griping device mounted to a patella using a circular elastic wire that biases one or more pins into a patella, in accordance with a sixth embodiment of the disclosure;

FIG. 40 is a view from the tibia side of the knee of the device of FIG. 39;

FIG. 41 is a partial perspective view of the device of FIG. 39;

FIG. 42 is a front view of an exemplary patella griping device mounted to a patella using an externally tensioned wire that biases one or more pins into a patella, in accordance with a seventh embodiment of the disclosure;

FIG. 43 is perspective view of the device of FIG. 42;

FIG. 44 is front view of an exemplary patella griping device mounted to a patella using an internal tensioning screw to tighten a circular wire that biases one or more pins into a patella, in accordance with an eighth embodiment of the disclosure;

FIG. 45 is a magnified view of the device of FIG. 44 showing the wire;

FIG. 46 is a perspective view of the device of FIG. 44;

FIG. 47 is an anterior view and a posterior view of a patella; and

FIG. 48 is an anterior view and a posterior view of a patella.

### DETAILED DESCRIPTION

Before describing in detail exemplary embodiments, it is noted that the embodiments reside primarily in combinations of apparatus components and processing steps related to objection location monitoring. Accordingly, components have been represented where appropriate by conventional symbols in the drawings, showing only those specific details that are pertinent to understanding the embodiments so as not to obscure the disclosure with details that will be readily apparent to those of ordinary skill in the art having the benefit of the description herein. Like numbers refer to like elements throughout the description.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

In some embodiments described herein, the term “coupled,” “connected,” and the like, may be used herein to indicate a connection, although not necessarily directly, and may include wired and/or wireless connections.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Radar technology, used widely in various industries, utilizes pulsed radio waves or signals at various frequencies to determine or track the distance, movement, and/or speed of an object based on the return/reflection of the signal and its modified frequency. Therefore, an object travelling away from the radar source would return a longer wavelength, and an object travelling towards the source would return a shorter wavelength. There are current radar applications available in the automobile and defense industries that aim to achieve fairly precise location determination. For example, there is at least one commercially available radar system, operating at 77 GHZ with wide 4 GHZ bandwidth, that allows for high resolution and accuracy with the use of Frequency-Modulated Continuous Wave (FMCW). However, the inventors are not aware of any conventional systems currently available that can achieve a resolution below one millimeter at short range, for example, at a range of 30 feet or less, such as 20 feet, 10 feet, five feet, four feet, three feet, or two feet, among others.

In an operating room (OR), a radar-based system having multiple radio wave sources can be used to triangulate or trilaterate the location of an object, especially an object that returns or reflects radio waves more efficiently than the surrounding objects, or that actively emits radio waves. Furthermore, varying the frequency of the waves emitted by the radio wave sources can allow the determination of the position or location of the object to be more accurate. If the object's positional accuracy is required to be sub-millimeter, waves of multiple wavelengths may be needed to determine the location and avoid misjudgment of the object's location, which may be in between the waves of a given wavelength(s). Submillimeter is defined as 1 μm-1 mm accuracy (micrometer-millimeter).

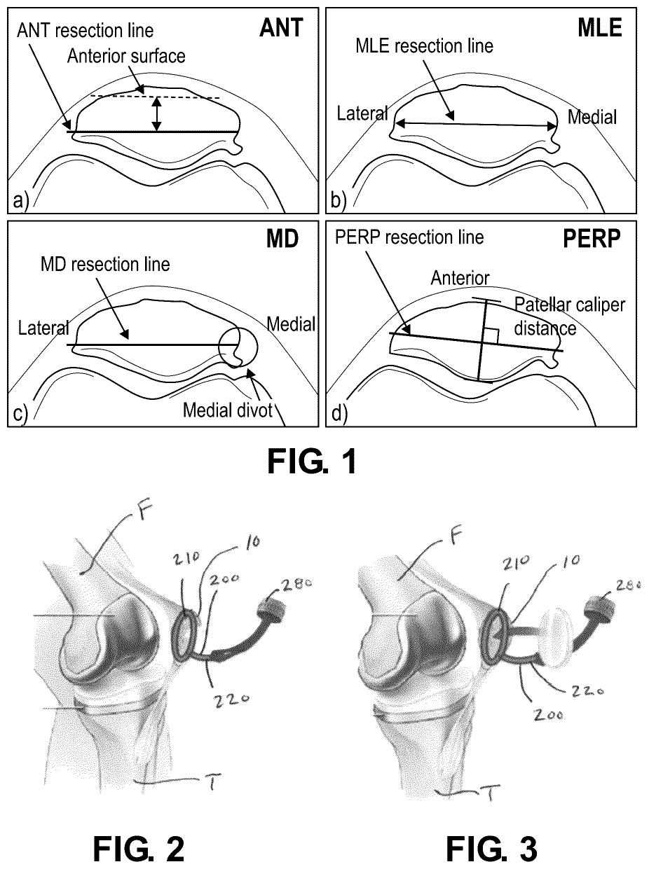

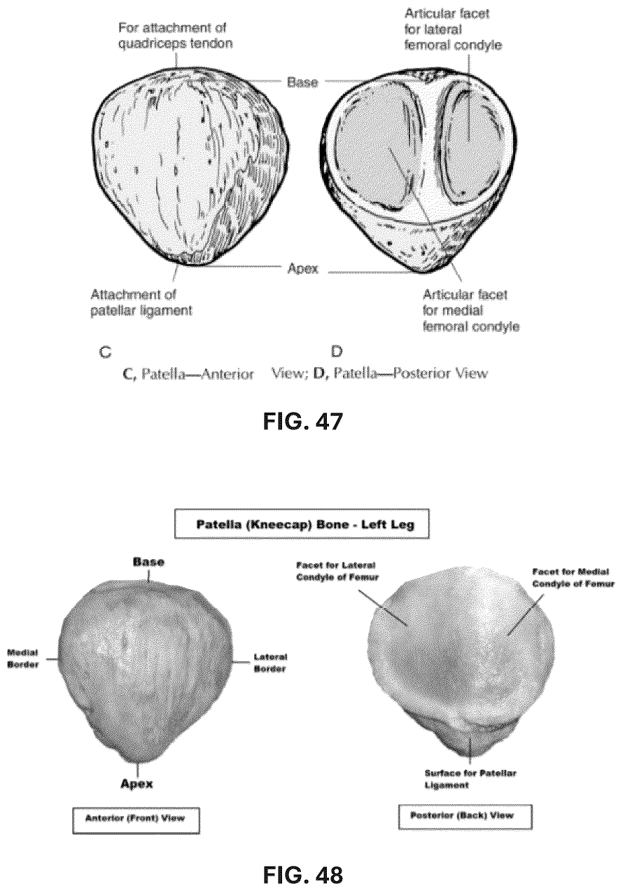

Referring now to the drawing figures, in which like elements are referred to by like reference numerals, FIG. 1 illustrates four radiographic images showing the desired resection (surgical removal) lines used in patella resurfacing: ANT—anterior surface; MLE—medial-lateral extents; MD—medial-divot; and PERP—perpendicular to the patellar caliper distance. A resected surface of the patella is a surface that is created by resection (removal) of part of the patella. FIGS. 47 and 48 show an example of a patella.

FIGS. 2 and 3 show an example of a device 200 (e.g., a mounting device) which is designed or configured to attach a tracking beacon 280 to a patella 10, which may have been resurfaced, and implants on a femur F and a tibia T. In this example, a ring-shape, circular-shaped, oval-shaped or other similarly shaped patella attachment 210 is attached to the patella 10, for example, to the resurfaced area of the patella 10. An extension, or extension arm, 220 extends from the patella attachment 210 to provide a mounting area for the beacon 280 outside of the patient, e.g., outside of the incision made in the skin of the patient at the patient's knee in this example.

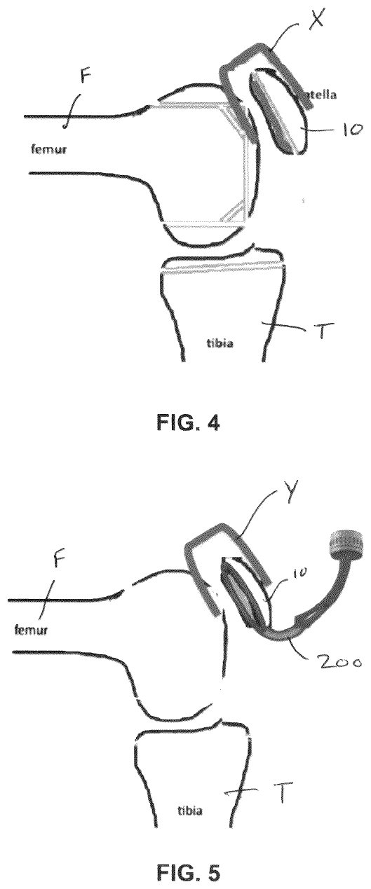

FIG. 4 shows a knee in a bent position and a pre-trial patellofemoral offset X prior to resurfacing of the patella 10. FIG. 5 shows the knee of FIG. 4 after resurfacing of the patella 10 and attachment of the device 200 to the patella 10, with a post-trial patellofemoral offset of Y. This arrangement can track the movement of the undersurface of the patella 10 relative to the femur F after the patella 10 has been resurfaced to provide a flat attachment surface for the device 200 (e.g., for the patella attachment 210 of the device 200).

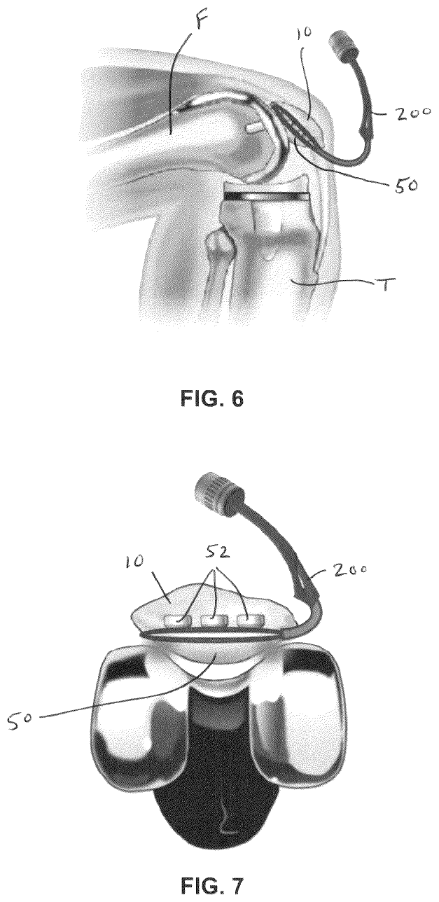

FIGS. 6 and 7 show a, for example, plastic button 50 attached to the patella 10 by way of a plurality of pegs 52. The example of an embodiment of a device 200 shown in FIGS. 2-7 is appropriate for a patella that has been resurfaced. It is important to note that not every patient needs the patella resurfaced. As a result, the example of the device 200 shown in FIGS. 2-7 is not appropriate for all situations.

Embodiments of the disclosure provide a number of different ways to attach a device 200 (e.g., a mounting device for a beacon) to a patella that has not been resurfaced by attaching to the patella at one or more areas of the patella that will not be removed if/when the patella is resurfaced. Such embodiments provide for the rigid attachment of the mounting device (e.g., 200) to the patella prior to resection, and for the device to remain in place during and after resection of the patella and insertion of the trial implant(s). This allows the device, and thus the tracking beacon (e.g., 280), to remain in place through all range of motion (ROM) and kinematic assessments with the patella reduced and tracking in the trochlear groove of the femur.

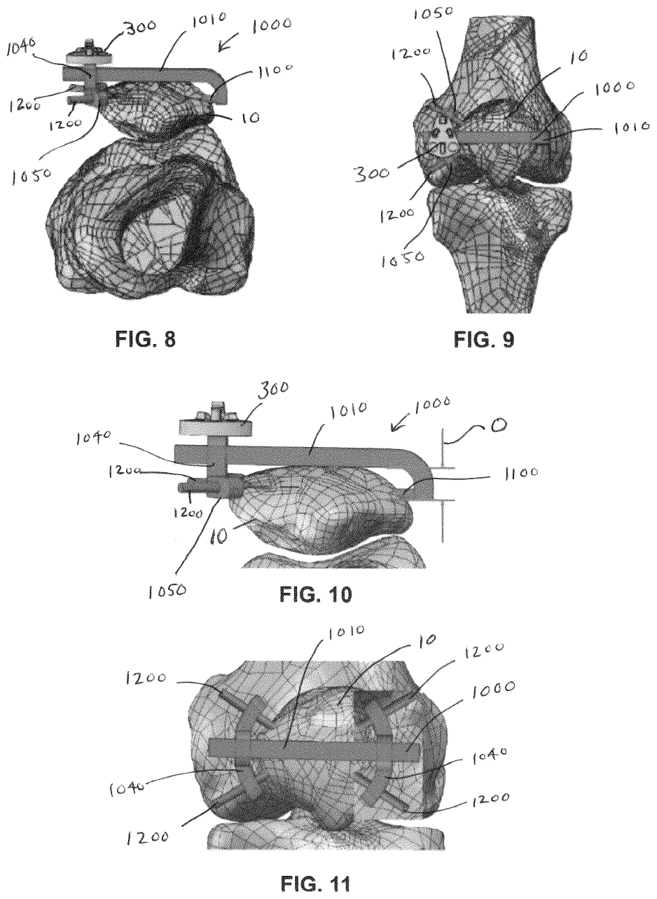

FIGS. 8-46 show multiple embodiments of devices for attaching a tracking beacon to a patella without contacting the portion(s) of the patella that will be removed if resurfacing of the patella is needed. In FIGS. 8-46, the tracking beacon (e.g., 280) is omitted to allow a clearer depiction of the devices. In many of the figures, an exemplary tracking beacon mount, or anchor, is shown and represents the mounting location of the tracking beacon. In FIGS. 8-46, the device (e.g., 200) for attaching a tracking beacon to a patella may be referred to as the “patella attachment,” in part to clearly differentiate the embodiments of FIGS. 2-7 from the embodiments of FIGS. 8-46.

FIGS. 8-11 show examples of a first embodiment of a device for attaching a tracking beacon to a patella without contacting the portion of the patella that will be removed if resurfacing of the patella is needed. In the first embodiment, a beacon mount, for example, an anchor, 300 is attached to the patella 10 by an embodiment of a patella attachment 1000 that has a number of pins that result in lateral fixation of a main member 1010 to the patella 10. In the example shown in FIGS. 8-10, a fixed pin 1100 is located at a first end of the main member 1010, and two movable pins 1200 are located at a second end of the main member 1010 opposite to the first end. The oblique relative location of the two movable pins 1200 help prevent the patella attachment 1000 from rotating around, or otherwise moving relative to, the fixed pin 1100. In embodiments, the fixed pin 1100 and the movable pins 1200 are inserted between 3 and 5 mm into the bone. The movable pins 1200 can be secured in position in pin holding portions 1050 by, for example, friction or a set screw or other mechanical securing means or method. In this example, the anchor 300 is attached to a sliding portion 1040 that can slide relative to the main member 1010 to adjust for different size patellas. The sliding portion 1040 can be secured to the main member 1010 by one or more set screws or other mechanical securing means or methods. In embodiments, an offset O of the fixed pin 1100 relative to the main member 1010 is, for example, 9 mm to accommodate a minimum of 10 mm bone thickness. Other embodiments may have a smaller or larger offset O. The main member 1010 can include a threaded connection (not shown in FIGS. 8-10) similar to a turnbuckle to provide lateral compression.

FIG. 11 shows an alternate configuration of the first embodiment that includes two additional movable pins 1200 at an end opposite to the moveable pins 1200 (i.e., on the right-hand end in FIG. 11) of the configuration shown in FIGS. 8-10. In the configuration shown in FIG. 11, four of the movable pins 1200 are used, whereas in the configuration shown in FIGS. 8-10 only two of the movable pins 1200 are used. In one implementation, the configuration shown in FIG. 11 replaces the fixed pin 1100 shown in FIGS. 8-10 with the two additional movable pins 1200 shown on the right-hand side in FIG. 11. In another implementation, the configuration shown in FIG. 11 can continue to include the fixed pin 1100 in addition to the two additional movable pins 1200 shown on the right-hand side in FIG. 11. Although they are omitted from FIG. 11 for clarity, the other features of the first embodiment are also applicable to the configuration shown in FIG. 11.

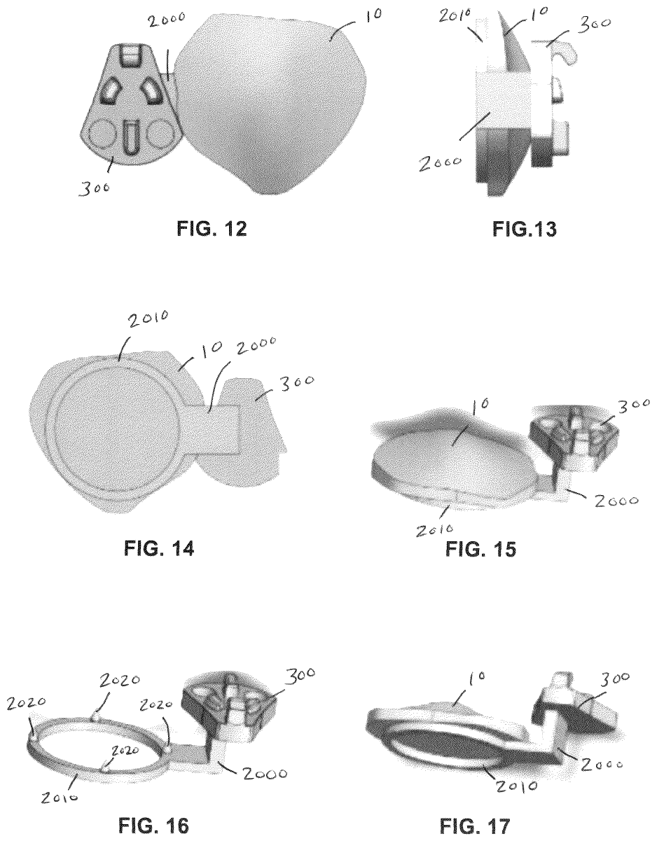

FIGS. 12-17 show an example of a second embodiment of the device for attaching a tracking beacon to a patella that has been resurfaced by friction fixation. In the second embodiment, the anchor 300 is attached to the patella 10 by a device or attachment 2000 having a number of spikes 2020 that penetrate a resurfaced underside of the patella 10. In this example, the spikes 2020 extend from a ring (or other shaped member) 2010 that is sized slightly smaller than the resurface underside of the patella 10. Different size rings 2010 can be used for different size patellas. In various embodiments, the ring 2010 may be round, oval, polygon or another shape that fits within the area of the resurface underside of the patella 10. In embodiments, the ring 2010 is attached to the patella 10 before a trial implant, button, or the like is attached to the patella 10. In embodiments, the ring 2010 is attached to the patella 10 after the trial implant is attached to the patella 10. In embodiments, the ring 2010 is incorporated into the trial implant (not shown).

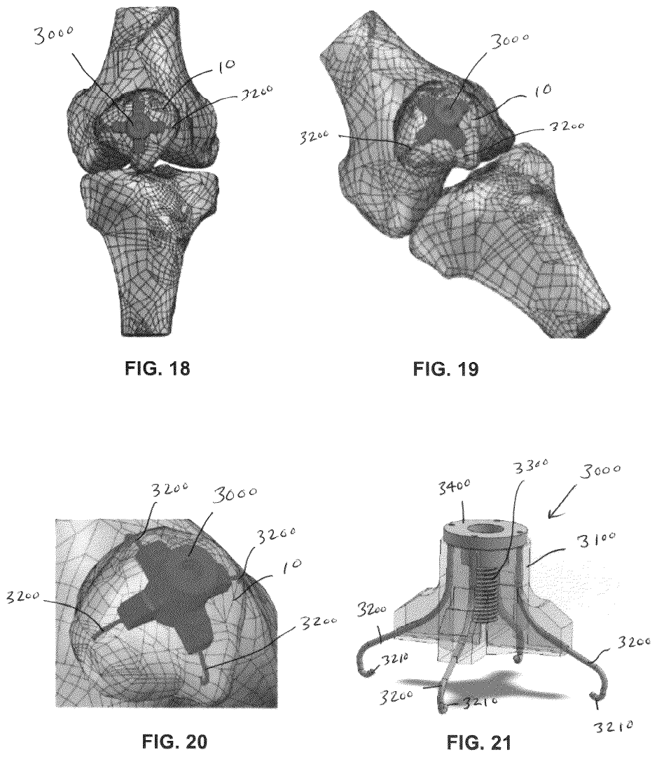

FIGS. 18-21 show an example of a third embodiment of the device for attaching a tracking beacon to a patella, with this one including a central tightening device. In the third embodiment, the anchor 300 (not shown) is attached to the patella 10 by an embodiment of a patella attachment 3000 having a plurality of hooks 3210 located on the ends of legs 3200. Each of the legs 3200 passes through a passage 3110 in a main body 3100 and is fixed to a member, in this example an upper ring 3400, that is movable relative to the main body 3100. The hooks 3210 are placed around the edges of the patella 10, (for example, while the upper ring 3400 is pushed down so that the legs 3200 extend farther outside of the passages 3110), and then the movable member, e.g., the upper ring 3400, is moved upward relative to the main body 3100. This upward movement of the movable-member upper ring 3400 pulls the legs 3200 upward, resulting in the hooks 3210 being drawn inward and exerting a gripping force on, and/or digging into, the patella 10. While this illustrated example includes four legs 3200, other examples include three legs 3200 or five or more legs 3200. In this example, the upper ring 3400 is biased upward relative to the main body 3100 by a spring 3300 within the main body 3100. FIG. 21 shows the upper ring 3400 in the downward-most position in which the underside of the upper ring 3400 contacts a top end of the main body 3100. In this example, this position is achieved by pressing the upper ring 3400 downward toward the main body 3100. In this position, the legs 3200 are in an extended position that, in some cases, allows the hooks 3210 to be positioned around the patella 10 without manually spreading the ends of the legs 3200 apart. Releasing downward pressure on the upper ring 3400 allows the spring 3300 to move the upper ring 3400 away from the main body 3100 and thereby pull the legs 3200 inward to grip the patella 10. In embodiments, a screw drive (not shown) is provided in addition to, or instead of, the spring 3300 to allow the surgeon to create tension (or additional tension) in the legs 3200.

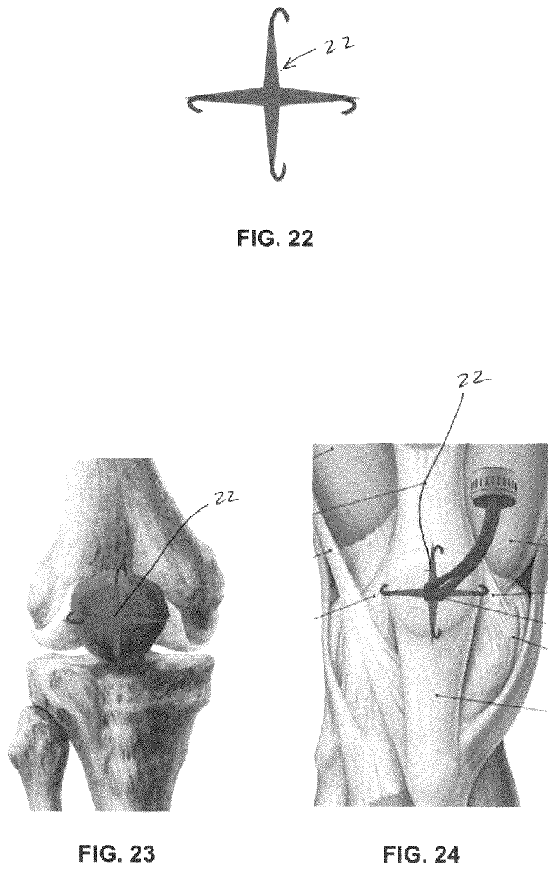

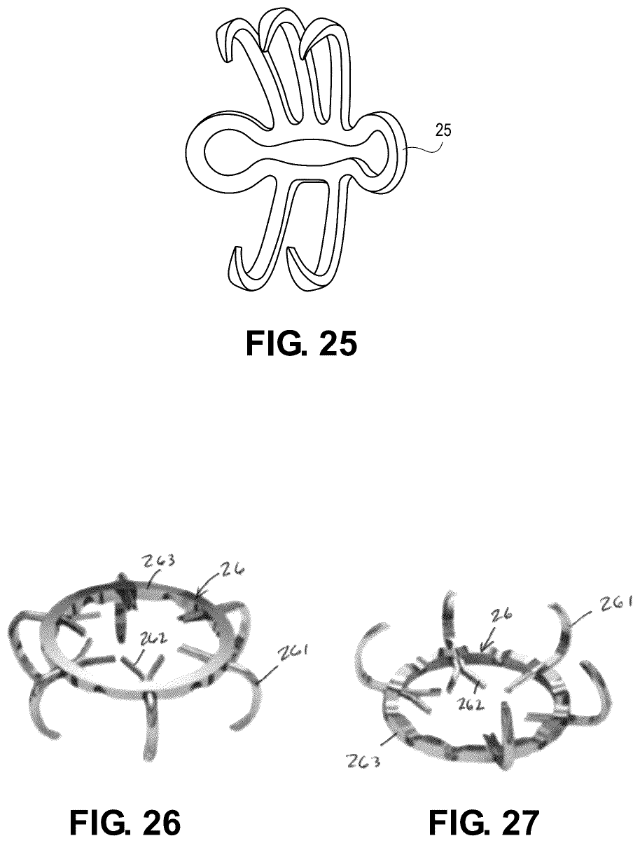

FIGS. 22-27 show various examples of tracking beacon attachment devices that grip the patella with a plurality of hooks. FIGS. 22-24 show a device 22 having four resilient legs that attach to the patella using resilience or spring force, where the material that makes up the device 22 is a material (e.g., a plastic, a polymer, or a springy metal) that, when loaded (e.g., pushed down), bends or deforms elastically to fit around a patella 10, and then returns to its original shape when onloaded, such that the device 22 exerts a gripping force on the patella 10. FIG. 25 shows a device 25 having six legs that attach to the patella using resilience or spring force. FIGS. 26 and 27 show a device 26 having, in this example, five or six legs 261 that are each independently biased inward by two resilient leg extensions 262 acting on a ring portion 263. The leg extensions 262 are made of a resilient material, such as plastic, a polymer, or a springy metal, that the ring portion 263 deforms elastically from the “V” shape, where the material's elastic force tends to pull the legs 261 toward the center of the ring portion 263 as the leg extensions 262 seek to elastically return to their “V” shape. When one of the legs 261 is pulled radially outward to extend around the patella, the leg extension 262 elastically presses against a recess or hole in the ring portion 263 and thereby exert a force on the ring portion 263 that biases the leg 261 radially inward. This inward bias exerts a gripping force on the patella.

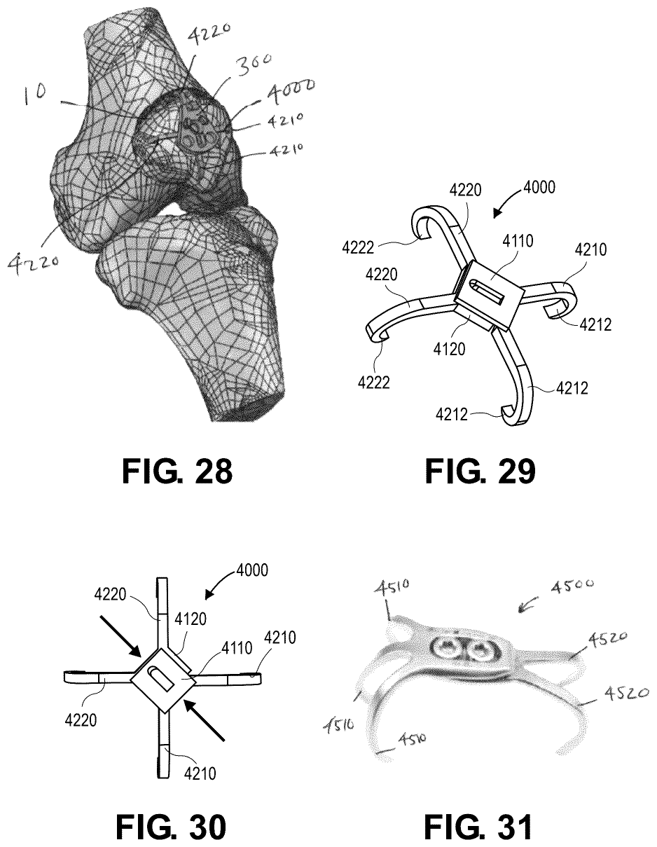

FIGS. 28-31 show examples of a fourth embodiment of the device for attaching a tracking beacon to a patella, where the device includes a sliding clamp. In the example shown in FIGS. 28-30, the anchor 300 is attached to the patella 10 by an embodiment of a patella attachment 4000 having a plurality of hooks 4212, 4222 located on the ends of legs 4210, 4220. As shown in FIGS. 29 and 30, the device 4000 has a first plate 4110 fixed to two legs 4210 that slides relative to a second plate 4120 that is fixed to two legs 4220. In embodiments, the first plate 4110 is selectably slidable relative to the second plate 4120 between a first position (e.g., open position) and a gripping position. In embodiments, a screw, a nut and bolt, or the like is provided such that the two plates 4110, 4120 are configured to, and can be, fixed relative to each other after that the hooks (first hooks) 4212 of the legs (first legs) 4210 and the hooks (second hooks) 4222 of the legs (second legs) 4220 are positioned to grip the patella in the gripping position. In some embodiments, the second plate 4120 has a threaded hole and a screw passes through a slot the first plate 4110 and into the threaded hole to clamp the two plates to each other when the screw is tightened. When the screw is loosened, the plates can move relative to each other as confined by the slot. In some embodiments one screw attaches the anchor 300 and the two plates to each other. In some embodiments, both the first plate 4110 and the second plate 4120 include slots (see, e.g. FIG. 30), and a nut and bolt are used to selectably fix or unfix the plates relative to each other, in a manner similar to that just described for the screw-based embodiment.

As the plates 4110, 4120 are pushed inward, the plates 4110, 4120 slide relative to each other and the legs 4210, 4220 move inward and the hooks 4212, 4222 press on, or penetrate, the patella, positionally fixing the patella attachment 4000 (and thus the anchor 300 and any tracking beacon that is attached to the anchor 300) to the patella. FIG. 31 shows an example of a device 4500 of the fourth embodiment having three legs 4510 and two legs 4520.

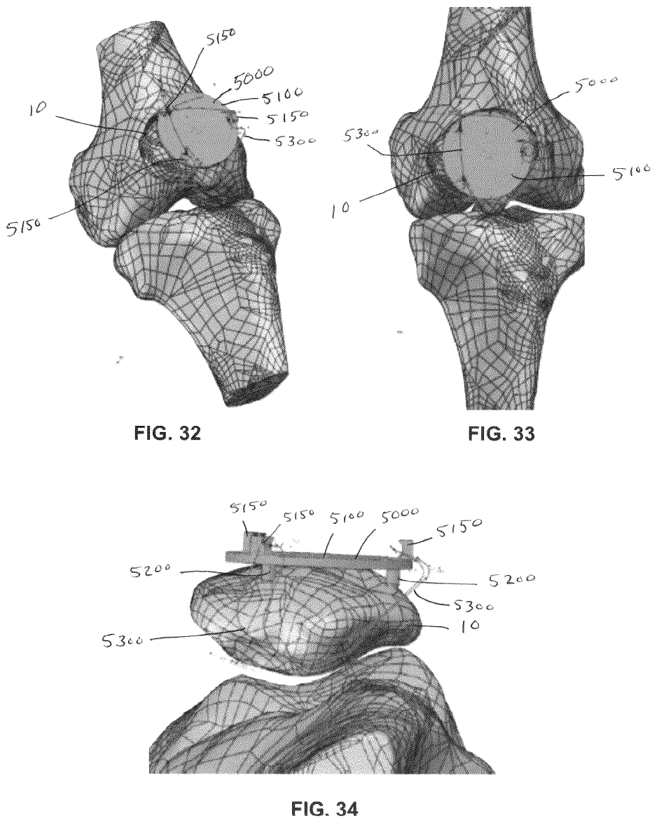

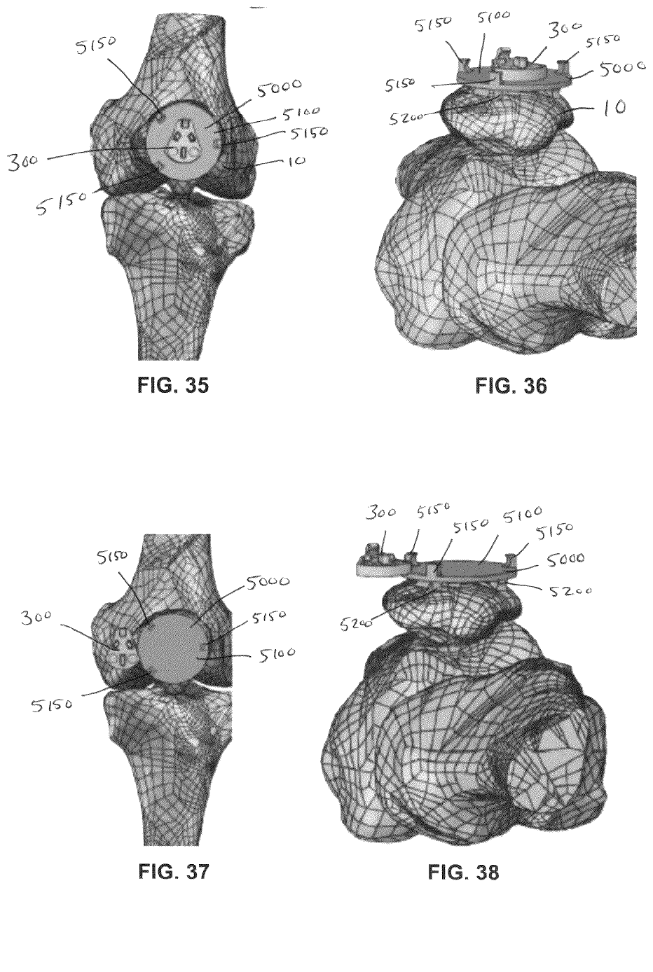

FIGS. 32-38 show an example of a fifth embodiment of the device for attaching a tracking beacon to a patella, where the device includes a wire or cabling. In the example shown in FIGS. 32-38, the anchor 300 is attached to the patella 10 by an embodiment of a patella attachment 5000 that includes a flexible member 5300, which may be a wire-shaped or wirelike member (e.g., a length of wire, such as stainless steel wire, a monofilament polymer line, a Kevlar string, etc.) that is wrapped around the patella 10. The device 5000 has a main member, in this example, a round plate 5100, that has a plurality of legs 5200 that extend downward toward the patella 10. A plurality of (in this example, three) wire retention tabs 5150 extend upward from an upper surface of the plate 5100. A wire 5300 or the like (e.g., wire-shaped member) is wrapped around the patella 10 and attached to the wire retention tabs 5150 to secure the plate 5100 to the patella 10. The wire 5300 can be tightened and attached to the tabs 5150 using pliers or another tool. The path of the wire 5300 is only one example of possible paths of the wire 5300. Also, one piece of the wire 5300 or multiple pieces of the wire 5300 can be used. FIGS. 35 and 36 show an exemplary positioning of the anchor 300 in a central location on the plate 5100. FIGS. 37 and 38 show an exemplary positioning of the anchor 300 in a location at the edge of the plate 5100. Other anchor locations can also be used. The location of the anchor can be determined based on the location of an incision so that the anchor is located outside of the patient's body.

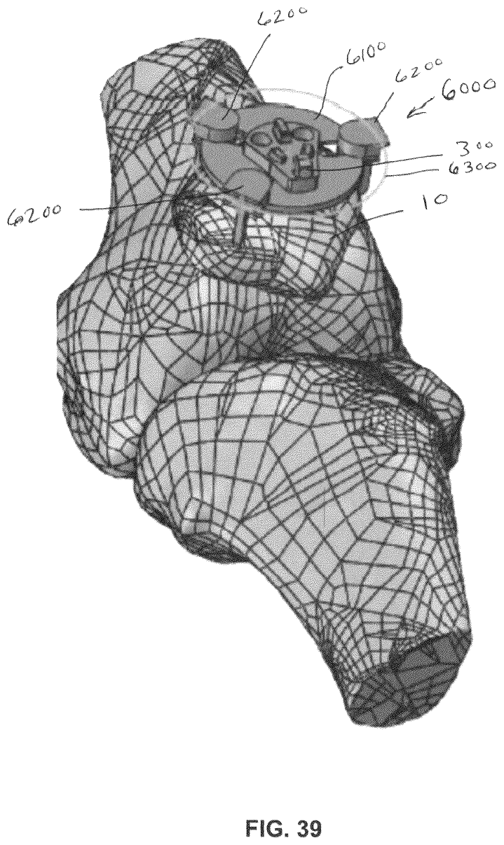

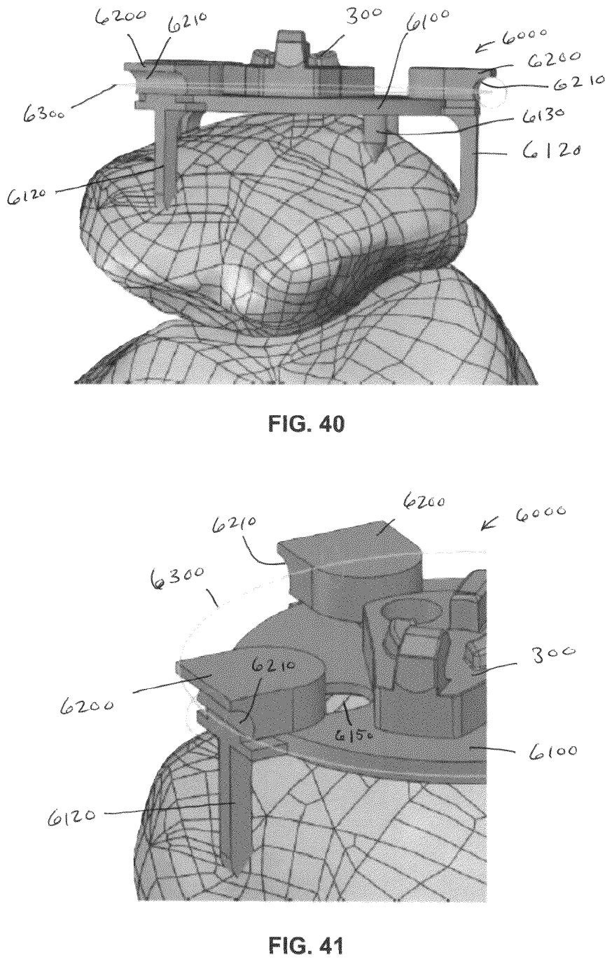

FIGS. 39-41 show an example of a sixth embodiment of the device for attaching a tracking beacon to a patella, where the device includes pins biased toward the patella by an elastic wire or cable or the like. In the example shown in FIGS. 39-41, the anchor 300 is attached to the patella 10 by a device 6000 that has, in this example, three sliding members 6200 that independently slide radially in slots 6150 in a plate 6100. Each of the sliding members 6200 has a leg 6120 that is fixed to the sliding member 6200 and extends downward below the plate 6100. As shown in FIG. 40, a number of pins 6130 extend downward from the plate 6100 and are configured to contact the patella 10. An elastic member such as, for example, an elastic wire-shaped member such as, for example, an elastic wire or cable, 6300 extends around the sliding members 6200 and applies a radially inward biasing force on the sliding members 6200. In this example, each sliding member 6200 has a recess 6210 configured to receive the wire 6300. The radially inward force on the sliding members 6200 biases the legs 6120 inward to grip the patella 10. The plate 6100 is configured to receive the anchor 300 such that the anchor 300 is positionally fixed relative to the plate 6100. Embodiments provide a uniform inward force on the legs 6120 without requiring unform displacement of the legs 6120, which provides secure attachment to the patella 10.

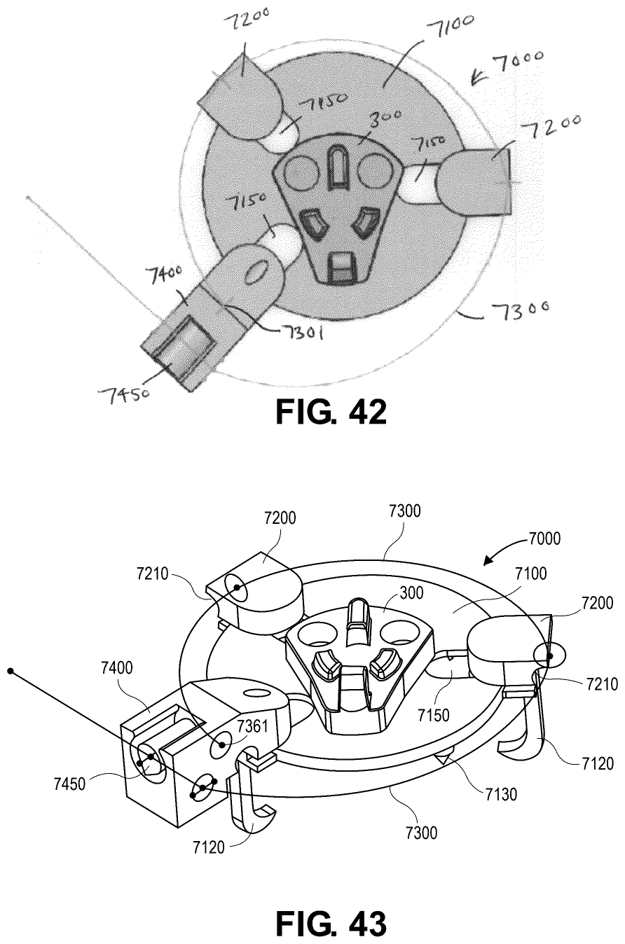

FIGS. 42-43 show an example of a seventh embodiment of the disclosure for attaching a tracking beacon to a patella using pins biased toward the patella by an externally tightened wire or cable. In the example shown in FIGS. 42-43, the anchor 300 is attached to the patella 10 by a device 7000 that has, in this example, two sliding members 7200 and one sliding member 7400 that independently slide radially in slots 7150 in a plate 7100. Each of the sliding members 7200, 7400 has a leg 7120 that is fixed to the sliding member 7200, 7400 and that extends downward below the plate 7100. As shown in FIG. 43, a number of pins 7130 extend downward from the plate 7100 and are configured to contact the patella 10. A wire-shaped member such as, for example, a wire or cable, 7300 extends around the sliding members 7200, 7400 and applies a radially inward biasing force on the sliding members 7200, 7400. In this example, each sliding member 7200 has a recess 7210 configured to receive the wire 7300. The sliding member 7400 is fixed to an end 7301 of the wire 7300 and has a mechanism 7450 that holds locks the wire 7300 in a clamped position. The radially inward force on the sliding members 7200, 7400 biases the legs 7120 inward to grip the patella 10. The plate 7100 is configured to receive the anchor 300 such that the anchor 300 is positionally fixed relative to the plate 7100. Embodiments provide a uniform inward force on the legs 7120 without requiring unform displacement of the legs 7120, which provides secure attachment to the patella 10.

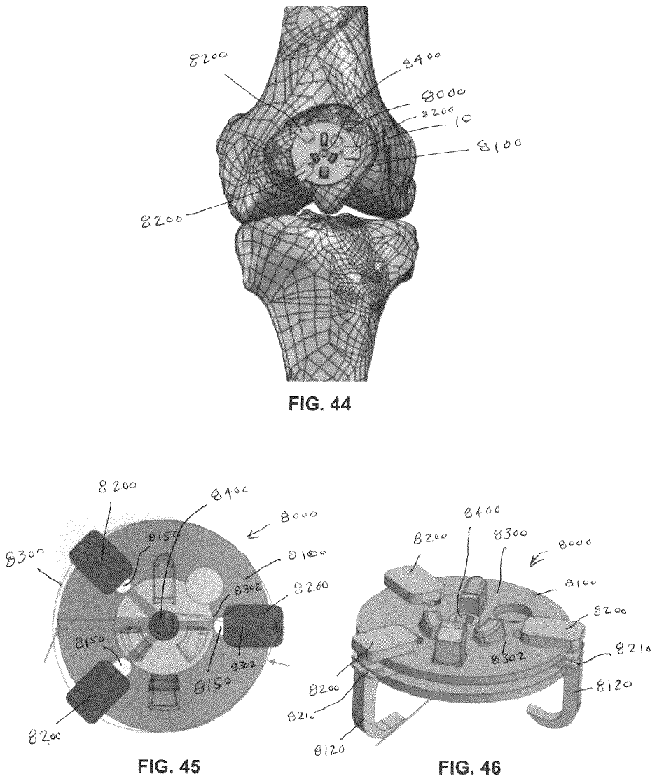

FIGS. 44-46 show an example of an eighth embodiment of the disclosure for attaching a tracking beacon to a patella using pins biased toward the patella by an internally tensioned wire or cable. In the example shown in FIGS. 44-46, the anchor 300 is attached to the patella 10 by a device 8000 that has, in this example, three sliding members 8200 that independently slide radially in slots 8150 in a plate 8100. Each of the sliding members 8200 has a leg 8120 that is fixed to the sliding member 8200 and that extends downward below the plate 8100. A number of pins 8130 (not shown) extend downward from the plate 8100 and are configured to contact the patella 10. A wire-shaped member such as, for example, a wire or cable, 8300 extends around the sliding members 8200 and applies a radially inward biasing force on the sliding members 8200. In this example, each sliding member 8200 has a recess 8210 configured to receive the wire 8300. In this example, the recesses 8210 are located in a portion of the sliding members 8200 that is below an upper surface of the plate 8100. Both ends 8302 of the wire 8300 are attached to a tensioning member such as, for example, a wire-shaped member tensioner (for example, a screw), 8400 located, in this example, in the center of the plate 8100. As best shown in FIG. 45, turning the screw 8400 (in this example, counterclockwise) will pull on the ends 8302 of the wire 8300 and tension the wire 8300 so as to apply a radially inward force of the sliding members 8200. The radially inward force on the sliding members 8200 biases the sliding members 8200, and thus the legs 8120, inward to grip the patella 10. In embodiments, the screw 8400 is positioned laterally such that it is accessed from an edge of the plate 8100. The plate 8100 is configured to receive the anchor 300 such that the anchor 300 is positionally fixed relative to the plate 8100. Embodiments provide a uniform inward force on the legs 8120 without requiring unform displacement of the legs 8120, which provides secure attachment to the patella 10.

As described above, particular embodiments of the disclosure provide secure attachment of a tracking beacon to a patella, or other bone, without requiring (or preventing) the resection or resurfacing of the patella. This is advantageous because the tracking beacon can be fixed to the patella once before assessments, resurfacing (if necessary), and placement and repositioning of a trial implant, thereby providing more efficient and precise placement of the permanent implant.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A mounting device for mounting a tracking beacon to a patella, comprising:
a beacon mount configured to receive the tracking beacon; and
a patella attachment that is configured to attach to an anterior surface of the patella,
wherein:
the patella attachment is configured to attach to the patella such that movement of the patella attachment is fixed relative to the patella,
the beacon mount is attached to the patella attachment such that movement of the beacon mount is fixed relative to the patella attachment, and
the patella attachment is configured to remain attached to the patella during movement of the patella.

2. The mounting device of claim 1, wherein the patella attachment comprises:
a main member that extends across the anterior surface of the patella;
a first pin attached to the main member and that is configured to be inserted into the patella; and
a movable second pin attached to the main member and that is configured to be inserted into the patella.

3. The mounting device of claim 1, wherein the patella attachment comprises:
a plurality of resilient legs, each of the legs having a hook that is configured to hook onto the patella;
a main body having passages configured to receive the legs such that the legs can move within the passages; and
a movable member attached to each of the legs,
wherein movement of the movable member causes the legs to move within the passages and move the hooks radially inward relative to the main body.

4. The mounting device of claim 1, wherein the patella attachment comprises:
a first plate;
a first leg fixed to the first plate, the first leg having a hook;
a second plate; and
a second leg fixed to the second plate, the second leg having a second hook,
wherein the first plate is selectably slidable relative to the second plate between a first position and a gripping position, and
the first plate and the second plate are configured to be fixed relative to each other such that the first hook of the first leg and the second hook of the second leg grip the patella in the gripping position.

5. The mounting device of claim 1, wherein the patella attachment comprises:
a plate configured to contact the patella; and
a wire-shaped member that is configured to wrap around the patella and attach to the plate to secure the plate to the patella.

6. The mounting device of claim 1, wherein the patella attachment comprises:
a plate configured to contact the patella;
a plurality of sliding members, each of the sliding members having a leg fixed to the sliding member; and
an elastic wire-shaped member configured to bias the sliding members radially inward such that the legs are biased toward the patella.

7. The mounting device of claim 1, wherein the patella attachment comprises:
a plate configured to contact the patella;
a plurality of sliding members, each of the sliding members having a leg fixed to the sliding member; and
a wire-shaped member configured to bias the sliding members radially inward such that the legs are biased toward the patella,
wherein one of the sliding members is configured to secure the wire-shaped member to the one of the sliding members.

8. The mounting device of claim 1, wherein the patella attachment comprises:
a plate configured to contact the patella;
a plurality of sliding members, each of the sliding members having a leg fixed to the sliding member;
a wire-shaped member configured to bias the sliding members radially inward such that the legs are biased toward the patella; and
a wire-shaped member tensioner configured to apply tension to ends of the wire-shaped member to bias the sliding members radially inward such that the legs are biased toward the patella.

9. A system for patella tracking, comprising:
the mounting device of claim 1;
a plurality of radio frequency transceivers, each of the plurality of radio frequency transceivers configured to emit a radio frequency signal at a first respective frequency;
a tracking beacon removably attachable to the mounting device, the tracking beacon being configured to modify the radio frequency signal from the respective first frequency to a second respective frequency different than the first respective frequency; and
a control device in communication with the plurality of radio frequency transceivers, the control device including processing circuitry configured to determine a location of the patella in three-dimensional space based at least in part on the modified radio frequency signal.

10. A mounting device for mounting a tracking beacon to a patella, comprising:
a beacon mount configured to receive a tracking beacon;
a patella attachment that is configured to attach to a resected surface of the patella; and
an extension that extends from the patella attachment and attaches the beacon mount to the patella attachment,
wherein:
the beacon mount is attached to the patella attachment via the extension such that movement of the beacon mount is fixed relative to the patella attachment,
the patella attachment is configured to remain attached to the patella during movement of the patella, and
the beacon mount is offset relative to the patella attachment by the extension.

11. A system for patella tracking, comprising:
the mounting device of claim 10;
a plurality of radio frequency transceivers, each of the plurality of radio frequency transceivers configured to emit a radio frequency signal at a first respective frequency;
a radio frequency beacon removably attachable to the mounting device, the radio frequency beacon being configured to modify the radio frequency signal from the respective first frequency to a second respective frequency different than the first respective frequency; and
a control device in communication with the plurality of radio frequency transceivers, the control device including processing circuitry configured to determine a location of the patella in three-dimensional space based at least in part on the modified radio frequency signal.

12. A system for patella tracking, comprising:
the mounting device of claim 1;
one or more optical fiducial markers attached to the mounting device or to the patella;
at least one image sensor configured to capture image frames of the one or more optical fiducial markers during movement of the patella; and
a control device including processing circuitry configured to:
identify the optical fiducial markers in the captured image frames using a computer-vision algorithm; and
compute a three-dimensional pose of the patella based on a spatial relationship of the optical fiducial markers.

13. The system of claim 12, wherein the one or more optical fiducial markers comprise passive, non-powered visual patterns selected from the group consisting of: a two-dimensional coded marker, a checkerboard pattern, a color-coded tag, and a geometric feature with known spacing.

14. The system of claim 12, wherein the at least one image sensor is selected from the group consisting of: a stereo camera pair, a structured-light depth camera, a time-of-flight sensor, and a handheld scanner with integrated optical tracking.

15. The system of claim 12, wherein the control device is configured to continuously track the patella in six degrees of freedom during knee flexion and extension by processing a sequence of the image frames.

16. The system of claim 12, wherein the optical fiducial markers are disposed on an extension that protrudes from the mounting device such that the optical fiducial markers remain in a line-of-sight of the image sensor throughout a knee range of motion.

17. The system of claim 12, wherein the control device is configured to detect partial or complete occlusion of the one or more optical fiducial markers and to maintain pose estimation through predictive motion modeling or interpolation based on previous ones of the image frames.

18. The system of claim 12, wherein the control device is configured to register the tracked patella pose to a pre-operative or intra-operative three-dimensional image of a knee to enable comparison of planned and actual patellar motion.

19. The system of claim 12, wherein the control device employs a trained neural network or other computer-vision model to automatically identify the one or more optical fiducial markers and compute the patella pose with sub-millimeter precision.

20. A method for intraoperative patella tracking, comprising:
attaching the mounting device of claim 1 to the patella;
positioning one or more optical fiducial markers on the mounting device;
capturing, by at least one image sensor, a sequence of image frames of the optical fiducial markers as a knee is moved through a range of motion; and
determining, by processing circuitry executing a computer-vision algorithm, a three-dimensional pose of the patella from a spatial configuration of the optical fiducial markers in the captured image frames.
